(19) United States
(12) Patent Application Publication
(10) Pub. No.: US 2026/0207056 A1
(43) Pub. Date: Jul. 23, 2026

(54) LOW-DOSE AGGREGATION-INDUCED EMISSION DYE BIOIMAGING DEVICE

(71) Applicant: THE CHINESE UNIVERSITY OF HONG KONG, SHENZHEN, Shenzhen (CN)

(72) Inventors: Zheng ZHAO, Shenzhen (CN); Benzhong TANG, Shenzhen (CN)

(73) Assignee: THE CHINESE UNIVERSITY OF HONG KONG, SHENZHEN, Shenzhen (CN)

(21) Appl. No.: 19/455,888

(22) Filed: Jan. 22, 2026

Related U.S. Application Data
(63) Continuation of application No. PCT/CN2025/138402, filed on Nov. 28, 2025.

(30) Foreign Application Priority Data
Jan. 20, 2025 (CN) 202510083885.4

(51) Int. Cl. A61B 5/00

(52) U.S. Cl. CPC A61B 5/0071

(57) ABSTRACT

The present disclosure provides a low-dose AIE dye bioimaging device, including an excitation light source, an imaging module, and a lock-in module. The excitation light source generates an excitation signal to make an organism produce frequency-matched fluorescence. The imaging module captures fluorescence signals from the organism and outputs them to the lock-in module, which controls the imaging module to capture fluorescence images periodically and processes them by a lock-in algorithm to obtain an amplitude value of each pixel periodically changing with the excitation signal. Convolving the amplitude signal of each pixel with a sine or cosine signal at the frequency of the excitation light source, together with the orthogonality of trigonometric functions, effectively suppresses noise, retains fluorescence signals that change at the frequency of the excitation light source. This greatly improves the SNR of fluorescence images, and enables clear observation of the fluorescence signals of low doses of AIE dyes.

### CROSS-REFERENCE TO RELATED APPLICATIONS

The application claims priority to Chinese patent application No. 202510083885.4, filed on Jan. 20, 2025, the entire contents of which are incorporated herein by reference.

### TECHNICAL FIELD

The present disclosure relates to the biomedical imaging field, and in particular to a low-dose aggregation-induced emission (AIE) dye bioimaging device based on lock-in technology.

### BACKGROUND

AIE dyes have attracted considerable attention in the medical field due to their long-term effective imaging ability in organisms. By enhancing fluorescence signals through molecular aggregation, AIE dyes can provide stable imaging signals, and are free of self-quenching that is common in traditional fluorescent dyes in a diluted state. Therefore, they can provide stable and clear fluorescence signals. However, this technology also has the following problems:

(I) For fluorescence imaging, high-dose AIE dyes need to be injected to obtain clear fluorescence images, while high-dose AIE dyes have unknown and potentially toxic effects on organisms and prolonged metabolic time, which further hinders their clinical application.

(II) Traditional excitation fluorescence imaging methods are affected by multiple factors, including focusing, light sources, optical filters, fluorescent probes, and samples. Especially in the biomedical imaging field, it is difficult to obtain clear fluorescence images in the long-wavelength band of 1,300 nm-1,700 nm, especially 1,400 nm-1,700 nm, preventing accurate observation and analysis of fine structures and dynamic changes in biological samples.

### SUMMARY

To address the shortcomings of the prior art, the present disclosure provides a low-dose AIE dye bioimaging device to solve one or more problems in the prior art.

In order to achieve the above purpose, the present disclosure provides the following technical solution:

The present disclosure proposes a low-dose AIE dye bioimaging device, including:
an excitation light source, which generates an excitation signal for test purpose to excite an organism under test, making the organism under test produce fluorescence at the same frequency as the excitation light source;
an imaging module, which captures fluorescence signals from the organism under test and outputs these signals to a lock-in module; and
a lock-in module, which controls the signal modulated by the excitation light source, controls the imaging module to capture fluorescence images at a fixed time interval, and applies a lock-in algorithm to process the fluorescence images to obtain an amplitude value of each pixel that changes periodically with the excitation signal.

Further, the lock-in module includes a calculation module and a signal generation module, in which:
the calculation module performs lock-in calculation on the captured fluorescence images; and
the signal generation module outputs square waves to modulate the excitation light source, making the excitation light source periodically modulate the organism under test.

Further, the excitation light source has a modulation frequency of 0.025-1,000 Hz and an output intensity of 1-726 mW/cm².

Further, a camera exposure time is set according to the modulation frequency and output intensity of the excitation light source, which ranges from 0.2 ms to 10s.

Further, the lock-in algorithm includes the following steps:
convolving the fluorescence signal components with a sine reference signal at the same frequency to obtain a sine component accumulation result;
convolving the fluorescence signal components with a cosine reference signal at the same frequency to obtain a cosine component accumulation result; and
calculating the amplitude value of each pixel that changes periodically with the excitation signal based on the sine component accumulation result and the cosine component accumulation result.

Further, convolving the fluorescence signal components with the sine reference signal at the same frequency involves a calculation formula as follows:

$${A}_{1} ( x , y ) = \frac{2}{kN} {\sum }_{i = 1}^{kN} f ( x , y , i ) sin ( \frac{2 π i}{N} )$$

where, A₁(x, y) represents the sine component accumulation result, f(x, y, i) represents the value of a pixel at the position (x, y) in the i th image, and

$$sin ( \frac{2 π i}{N} )$$

represents the sine reference signal.

Further, convolving the fluorescence signal components with the cosine reference signal at the same frequency involves a calculation formula as follows:

$${A}_{2} ( x , y ) = \frac{2}{kN} {\sum }_{i = 1}^{kN} f ( x , y , i ) cos ( \frac{2 π i}{N} )$$

where, A₂(x, y) represents the cosine component accumulation result, f(x, y, i) represents the value of a pixel at the position (x, y) in the i th image, and

$$cos ( \frac{2 π i}{N} )$$

represents the cosine reference signal.

Further, the amplitude value of each pixel that changes periodically with the excitation signal is calculated according to a calculation formula as follows:

$$A ( x , y ) = \sqrt{{{A}_{1} ( x , y )}^{2} + {{A}_{2} ( x , y )}^{2}}$$

where, A₁(x, y) represents the sine component accumulation result, A₂(x, y) represents the cosine component accumulation result, and A(x, y) represents the amplitude value of each pixel that changes periodically with the excitation signal.

Compared with the prior art, the present disclosure has the following beneficial effects:

Convolving the amplitude signal of each pixel in each fluorescence image with the sine or cosine signal at the same frequency as the excitation light source, together with the orthogonality of trigonometric functions, effectively suppresses noise, enabling retention of fluorescence signals that change at the same frequency as the excitation light source. This method significantly improves the signal-to-noise ratio (SNR) of fluorescence images, making it possible to clearly observe the fluorescence signals of the AIE dyes even at low doses. This not only lowers the experimental cost, but also reduces the impact of the dyes on organisms, facilitating safer and more environmentally friendly biological research and medical diagnosis.

Further, due to the improvement in SNR, the imaging quality of the fluorescence images is significantly enhanced. The fluorescence signals in the images become clearer and sharper, and the background noise is reduced. This enables researchers to identify and locate the sources of fluorescence signals more accurately, thereby improving the accuracy and reliability of biological research and medical diagnosis.

Further, convolution not only suppresses noise but also enhances the contrast between the fluorescent signals and the background. This increases the sensitivity for detecting fluorescence signals at low doses, enabling even extremely faint fluorescence signals to be clearly captured and displayed.

Due to the improvement in SNR and imaging quality, researchers can obtain clear fluorescence images while using lower doses of AIE dyes. This not only lowers the experimental costs, but also reduces the potential impact of the dyes on organisms, facilitating safer and more environmentally friendly biological research and medical diagnosis.

### BRIEF DESCRIPTION OF DRAWINGS

FIG. 1 is a schematic diagram of an imaging method of a low-dose AIE dye bioimaging device according to an embodiment of the present disclosure;

FIG. 2 is a structural schematic diagram of a low-dose AIE dye bioimaging device according to an embodiment of the present disclosure;

FIG. 3 is a structural schematic diagram of a lock-in module in a low-dose AIE dye bioimaging device according to an embodiment of the present disclosure;

FIG. 4 is a schematic diagram comparing an image obtained by a low-dose AIE dye bioimaging device according to an embodiment of the present disclosure with an image obtained by a traditional method;

Reference numerals: 1. organism under test; 2. imaging module; 3. lock-in module; 4. computer; 5. excitation light source.

### DETAILED DESCRIPTION OF THE EMBODIMENTS

In order to make the purpose, technical solution, and advantages of the present disclosure clearer, a low-dose AIE dye bioimaging device provided in the present disclosure will be further described in detail below with reference to attached figures and embodiments. The following description will make the advantages and features of the present disclosure clearer. It should be noted that the figures are very simplified and based on non-precise scales. They are only used to conveniently and clearly assist in explaining the purpose of the embodiments of the present disclosure. In order to make the purposes, features, and advantages of the present disclosure more obvious and understandable, the figures are attached for reference. It should be noted that the structures, proportions, and sizes shown in the attached figures of the specification are only used to assist in explaining the content disclosed in the specification for understanding and reading by those skilled in the art, and are not conditions limiting the implementation of the present disclosure. Therefore, they have no technical substantive significance. Any modifications to the structures, changes in proportional relationship, and adjustments in size shall fall within the scope of the technical content disclosed in the present disclosure provided that they do not influence the effect and purpose of the present disclosure.

A low-dose AIE dye bioimaging device includes an imaging module 2, a lock-in module 3, a computer 4, and an excitation light source 5.

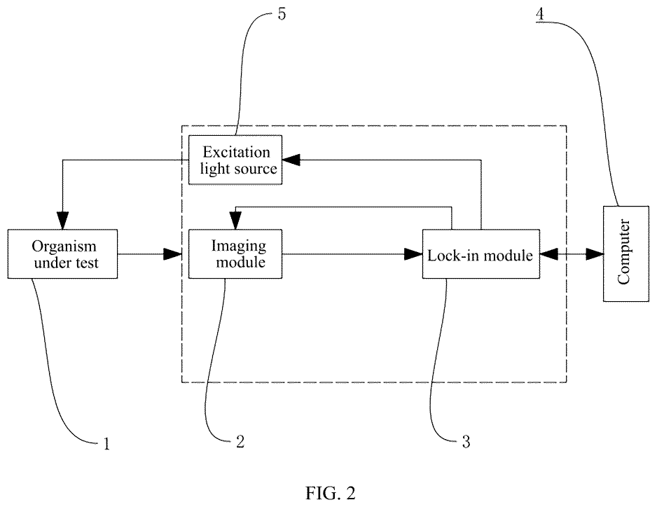

Specifically, as shown in FIG. 2, the excitation light source 5, located between an organism under test 1 and the lock-in module 3, generates an excitation signal for test purpose to excite the organism under test 1, making the organism under test 1 produce fluorescence at the same frequency as the excitation light source.

Further, in this embodiment, the excitation light source has a modulation frequency of 0.025 Hz and an output intensity of 32 mW/cm². A camera exposure time of 10 s is set for the imaging module 2 according to the modulation frequency and output intensity of the excitation light source. A camera sampling frequency (reciprocal of the exposure time) of the imaging module is obtained by multiplying the lock-in frequency by the number of images captured per cycle.

Further, the device also includes an imaging module 2, which captures fluorescence signals from the organism under test 1 and outputs these signals to the lock-in module 3. An output end of the imaging module 2 is connected to an input end of the lock-in module 3.

Specifically, in this embodiment, the imaging module 2 may be a high-sensitivity InGaAs or InSb camera to ensure that the imaging module 2 can still capture clear fluorescence signals under low-dose dye conditions.

Further, to achieve high sensitivity, the imaging module 2 can be provided with an image intensifier to enhance weak signals. The incident light passes through an objective lens, hits the photocathode of the image intensifier, and then is converted into an electron image by the photoelectric effect. The electron image continuously strikes a microchannel plate, ultimately generating a multiplication effect. The multiplication effect enhances the weak signals and re-excites a photon image, which is transmitted to the imaging module 2 for imaging. In this way, the imaging module 2 can still capture clear fluorescence images under low-dose dye conditions.

Further, the device also includes the lock-in module 3. The input end of the lock-in module 3 is connected to the output end of the imaging module 2. Specifically, the lock-in module controls the signal modulated by the excitation light source 5, synchronously obtains the fluorescence images captured by the imaging module 2, and applies a lock-in algorithm to process the fluorescence images to obtain an amplitude value of each pixel that changes periodically with the excitation signal.

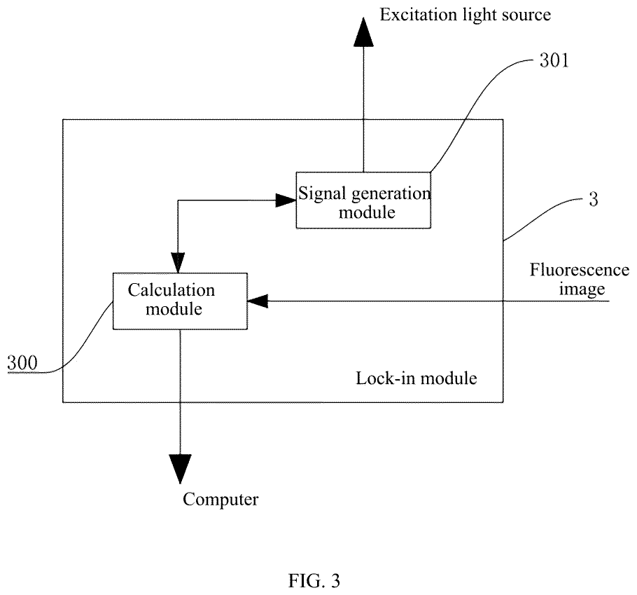

Specifically, as shown in FIG. 3, the lock-in module 3 includes a calculation module 300 and a signal generation module 301. The calculation module 300 and the signal generation module 301 are connected through a first interface. The calculation module 300 performs lock-in calculation on the captured fluorescence images (the specific lock-in calculation process is detailed in the method). The signal generation module 301 outputs square waves to modulate the excitation light source 5. The calculation module 300 receives a trigger signal or a synchronization signal from the signal generation module 301 through the first interface to ensure the synchronization between fluorescence image capture and light source excitation. Additionally, the calculation module 300 is also connected to the computer 4 through a second interface for outputting a lock-in calculation result to a display terminal (for example a display screen) of the computer 4 for display and analysis. Further, the calculation module 300 also receives the fluorescence images captured by the imaging module 2 through a third interface.

Further, the signal generation module 301 outputs a periodically changing square wave signal to modulate the excitation light source and excite the organism under test 1. The square wave signal collects N groups of data at equal intervals within one cycle. The data collected is used for analysis. Specifically, the data includes fluorescence intensity data, which reflects the emission intensity of the organism under test 1.

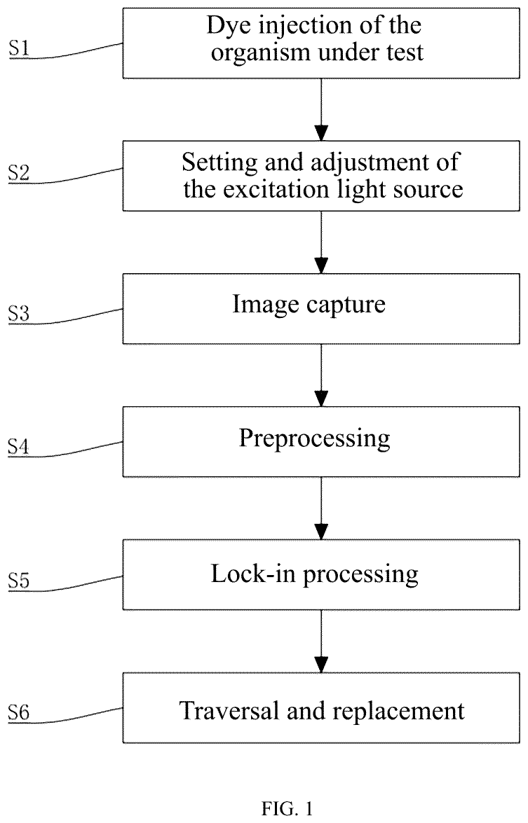

Accordingly, as shown in FIG. 1, for the low-dose AIE dye bioimaging device, the present disclosure provides an imaging method, which includes the following steps:

S1: selecting an organism for test and injecting a low dose of AIE dye. Specifically, select a suitable organism for test, for example a mouse. Then, narcotize, fix, or treat the organism under test 1 by other means to make it remain stable to reduce interference with test results.

S2: setting and adjusting an excitation light source: Select an excitation light source 5 with adjustable frequency to excite the organism under test. The periodically changing light signal generated by the excitation light source penetrates the skin of the mouse and excites molecules of the AILE dye pre-injected into the blood vessels, making these molecules emit fluorescence signals. The fluorescence signals include components at the same frequency as the excitation signal and components at other frequencies (such as background noise). Specifically, in the lock-in module 3, set the frequency and phase of the square wave signal through the signal generation module 301 to synchronize the lock-in module with the periodic change of the excitation light source. At the same time, set trigger conditions for image capture to ensure that N groups of fluorescence images can be captured after the excitation light source completes a periodic change.

S3: image capture. Capture the fluorescence signals from the organism under test at a fixed time interval. Specifically, when the signal generation module 301 in the lock-in module 3 generates a synchronization signal at the same frequency as the signal modulated by the excitation light source, the synchronization signal is transmitted to a trigger port of the imaging module 2. When the synchronization signal meets the preset trigger conditions, the imaging module 2, for example an InGaAs camera, captures the images. Specifically, the synchronization signal generated by the signal generation module 301 can serve as a trigger signal for a timer or a calculator. Therefore, when each synchronization signal arrives, a new time interval starts, and image capture is triggered for the imaging module 2 at the end of the time interval.

S4: preprocessing. Process the fluorescence signals and generate the fluorescence images. By applying an image processing algorithm or signal processing software, the imaging module 2 extracts fluorescence signal components at the same frequency as the excitation signal from the fluorescence signals, and recombines the fluorescence signal component data and noise suppression data into images to generate the fluorescence images.

S5: lock-in processing. Apply lock-in processing to the fluorescence images to separate the fluorescence signal components at the same frequency as the excitation light source from the fluorescence images and generate a lock-in imaging result. Specifically, the lock-in processing includes the following steps:

S500: convolving the fluorescence signal components with a sine reference signal at the same frequency to obtain a sine component accumulation result.

Convolving the fluorescence signal components with the sine reference signal at the same frequency involves a calculation formula as follows:

$${A}_{1} ( x , y ) = \frac{2}{kN} {\sum }_{i = 1}^{kN} f ( x , y , i ) sin ( \frac{2 π i}{N} ) \qquad ( 1 )$$

where, A₁(x, y) represents the sine component accumulation result, f(x, y, i) represents the value of a pixel at the position (x, y) in the i th image, and

$$sin ( \frac{2 π i}{N} )$$

represents the sine reference signal.

S501: convolving the fluorescence signal components with a cosine reference signal at the same frequency to obtain a cosine component accumulation result.

Convolving the fluorescence signal components with the cosine reference signal at the same frequency involves a calculation formula as follows:

$${A}_{2} ( x , y ) = \frac{2}{kN} {\sum }_{i = 1}^{kN} f ( x , y , i ) cos ( \frac{2 π i}{N} ) \qquad ( 1 )$$

where, A₂(x, y) represents the cosine component accumulation result, f(x, y, i) represents the value of a pixel at the position (x, y) in the i th image,

$$cos ( \frac{2 π i}{N} )$$

represents the cosine reference signal, and (x, y) represents the spatial position coordinates of a pixel.

S502: calculating the amplitude value of each pixel that changes periodically with the excitation signal based on the sine component accumulation result and the cosine component accumulation result. The amplitude value of each pixel that changes periodically with the excitation signal is calculated according to a calculation formula as follows:

$$A ( x , y ) = \sqrt{{{A}_{1} ( x , y )}^{2} + {{A}_{2} ( x , y )}^{2}}$$

where, A₁(x, y) represents the sine component accumulation result, A₂(x, y) represents the cosine component accumulation result, and A(x, y) represents the amplitude value of each pixel that changes periodically with the excitation signal.

During the above-mentioned lock-in processing, the orthogonality of trigonometric functions can be used to separate intensities of fixed wavelength light components at the same frequency as the excitation signal and reject irregular fluctuations in measurement data caused by electronic component temperature drift, thermal noise, and environmental disturbances, thereby greatly improving the SNR of imaging. As the measurement duration, that is, the number of measurement cycles, increases, the SNR can be continuously improved.

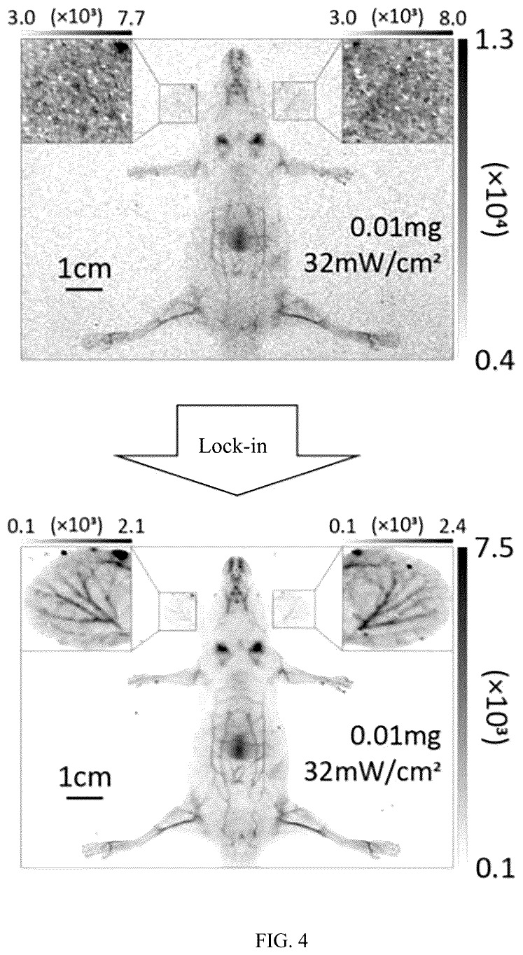

S6: traversal and replacement: Parallel calculation is performed for each pixel in each fluorescence image. Specifically, the amplitude value of each pixel that changes periodically with the excitation signal after convolution is used to replace the original amplitude signal of the corresponding pixel in the fluorescence image. This replacement enhances the SNR of fluorescence imaging, and enables clear fluorescence imaging of AIE dyes at low doses. Finally, clear fluorescence images are output to the computer and displayed at the display terminal of the computer. As shown in FIG. 4, a comparison between a traditional imaging method and the lock-in imaging method provided in the present disclosure is intuitively shown. With an AIE dye dose of 0.01 mg and an excitation light power density of 32 m W/cm², the method provided in the present disclosure leads to a clearer blood vessel structure, morphology, branches, and connections of the mouse. Therefore, the structure and functional status of blood vessels can be explained based on the results of lock-in imaging.

The technical features of the above embodiments can be combined arbitrarily. For concise description, not all possible combinations of the technical features in the above embodiments are described. However, as long as there is no contradiction between the combinations of these technical features, they shall be considered to fall within the scope of this specification.

The above embodiments only express several embodiments of the present disclosure, which are described in more specific and detailed manners, but shall not be construed as a limitation on the scope of the present disclosure. It should be pointed out that ordinary persons skilled in the art can also make several modifications and improvements without departing from the concept of the present disclosure, which belong to the protection scope of the present disclosure. Therefore, the protection scope of the present disclosure shall be subject to the attached claims.

What is claimed is:

1. A low-dose AIE dye bioimaging device, comprising:
an excitation light source, which generates an excitation signal for test purpose to excite an organism under test, making the organism under test produce fluorescence at the same frequency as the excitation light source;
an imaging module, which captures fluorescence signals from the organism under test and outputs these signals to a lock-in module; and
a lock-in module, which controls the signal modulated by the excitation light source, controls the imaging module to capture fluorescence images at a fixed time interval, and applies a lock-in algorithm to process the fluorescence images to obtain an amplitude value of each pixel that changes periodically with the excitation signal.

2. The low-dose AIE dye bioimaging device according to claim 1, wherein
the lock-in module comprises a calculation module and a signal generation module, wherein
the calculation module performs lock-in calculation on the captured fluorescence images; and
the signal generation module outputs square waves to modulate the excitation light source, making the excitation light source periodically modulate the organism under test.

3. The low-dose AIE dye bioimaging device according to claim 1, wherein the excitation light source has a modulation frequency of 0.025-1,000 Hz and an output intensity of 1-726 mW/cm².

4. The low-dose AIE dye bioimaging device according to claim 3, wherein a camera exposure time is set according to the modulation frequency and output intensity of the excitation light source, which ranges from 0.2 ms to 10 s.

5. The low-dose AIE dye bioimaging device according to claim 1, wherein the lock-in algorithm includes the following steps:
convolving the fluorescence signal components with a sine reference signal at the same frequency to obtain a sine component accumulation result;
convolving the fluorescence signal components with a cosine reference signal at the same frequency to obtain a cosine component accumulation result; and
calculating the amplitude value of each pixel that changes periodically with the excitation signal based on the sine component accumulation result and the cosine component accumulation result.

6. The low-dose AIE dye bioimaging device according to claim 5, wherein convolving the fluorescence signal components with the sine reference signal at the same frequency involves a calculation formula as follows:
$${A}_{1} ( x , y ) = \frac{2}{kN} {\sum }_{i = 1}^{kN} f ( x , y , i ) sin ( \frac{2 π i}{N} )$$
where, A₁(x, y) represents the sine component accumulation result, f(x, y, i) represents the value of a pixel at the position (x, y) in the i th image, and
$$sin ( \frac{2 π i}{N} )$$
represents the sine reference signal.

7. The low-dose AIE dye bioimaging device according to claim 5, wherein:
convolving the fluorescence signal components with the cosine reference signal at the same frequency involves a calculation formula as follows:
$${A}_{2} ( x , y ) = \frac{2}{kN} {\sum }_{i = 1}^{kN} f ( x , y , i ) cos ( \frac{2 π i}{N} )$$
where, A₂(x, y) represents the cosine component accumulation result, f(x, y, i) represents the value of a pixel at the position (x, y) in the i th image, and
$$cos ( \frac{2 π i}{N} )$$
represents the cosine reference signal.

8. The low-dose AIE dye bioimaging device according to claim 5, wherein the amplitude value of each pixel that changes periodically with the excitation signal is calculated according to a calculation formula as follows:
$$A ( x , y ) = \sqrt{{{A}_{1} ( x , y )}^{2} + {{A}_{2} ( x , y )}^{2}}$$
where, A₁(x, y) represents the sine component accumulation result, A₂(x, y) represents the cosine component accumulation result, and A(x, y) represents the amplitude value of each pixel that changes periodically with the excitation signal.
